(19) United States
(12) Patent Application Publication
(10) Pub. No.: US 2026/0199571 A1
(43) Pub. Date: Jul. 16, 2026

(54) DETERMINING INTERNAL FILTRATION RATE WITHIN A CAPILLARY HEMODIALYZER

(71) Applicant: GAMBRO LUNDIA AB, Lund (SE)

(72) Inventors: Danilo DONATO, Rende-Cosenza (IT); Markus STORR, Filderstadt (DE)

(21) Appl. No.: 19/560,778

(22) Filed: Mar. 9, 2026

Related U.S. Application Data
(63) Continuation of application No. 17/768,516, filed on Oct. 13, 2020.

(30) Foreign Application Priority Data
Oct. 14, 2019 (EP) 19202905.6

(51) Int. Cl. A61M 1/16

(52) U.S. Cl. CPC A61M 1/1605; A61M 1/1627; A61M 1/1647

(57) ABSTRACT

The present disclosure relates to a method and a device for determining an internal filtration rate IFR within a capillary hemodialyzer.

### CROSS-REFERENCE TO RELATED APPLICATIONS

This application is a continuation application of U.S. application Ser. No. 17/768,516, filed Apr. 13, 2022, which is a national stage entry under 35 U.S.C. § 371(b) of PCT International Application No. PCT/EP2020/078807, filed Oct. 13, 2020, which claims the benefit of European Patent Application Serial No. 19202905.6, filed on Oct. 14, 2019, the entire disclosures of all of which are incorporated herein by reference.

### TECHNICAL FIELD

The present disclosure relates to a method and a device for determining an internal filtration rate IFR within a capillary hemodialyzer.

### DESCRIPTION OF THE RELATED ART

The difficult removal of middle molecules from patients suffering from end-stage kidney disease represents one of the major challenges of hemodialysis. High concentrations of middle molecular weight (MW) solutes, such as β₂-microglobulin, myoglobin, complement factor D, and polyclonal free light chains (κ-FLC and λ-FLC), have been correlated to critical clinical outcomes in chronic kidney disease patients. In conventional hemodialysis, low MW solutes (e.g. urea and creatinine) are effectively removed by diffusion, whereas the removal of middle MW solutes is generally achieved with the superimposition of convective transport over pure diffusion. The use of more water-permeable high-flux dialyzers and hemodiafiltration to increase convective transport has been shown to enhance the clearance of middle MW solutes, but still leads to unsatisfactory clinical results.

In EP 3 102 312 B1 and EP 3 102 314 B1, a new class of membranes, defined as Medium cut-off (MCO), and hemodialyzers comprising such membranes are disclosed. The particular features of MCO membranes include high MW retention onset and MW cut-off value close to the molecular weight of albumin, allowing for the removal of solutes up to ca. 45 kDa with negligible albumin loss. The use of hemodialyzers equipped with MCO membranes has made it possible to perform a new therapy called expanded hemodialysis (HDx), in which convective and diffusive transport are efficiently combined to increase dialyzer removal capability over a wide range of molecular weights. This is due to the interplay among the larger permeability of the MCO membranes as compared to that of high-flux membranes, and their geometrical structure, which increase convective transport across the hemodialyzer membrane by enhancing internal filtration (i.e. movement of plasma water from the blood compartment towards the dialysate compartment across the porous membrane in the proximal part of the hemodialyzer) and back filtration (i.e. movement of plasma water from the dialysate compartment towards the blood compartment in the distal part of the hemodialyzer). The compensation of internal filtration (IF) with an adequate amount of back filtration (BF) also permits to avoid a complex set-up and the use of fluid reinfusion, thus overcoming some practical issues of hemodiafiltration.

Methods to experimentally quantify the rate of IF and BF applied to hemodialyzers equipped with MCO membranes have been recently described by A. Lorenzin et al.: “Quantification of internal filtration in hollow fiber hemodialyzers with Medium cut-off membrane”, Blood Purif. 46 (2018) 196-204. However, the experimental quantification of IF generally requires complex settings, and it is not cost-effective. Therefore, a simple and effective method for determining internal filtration rate IFR within a hemodialyzer is still lacking.

### SUMMARY

The present disclosure provides a method and a device for determining an internal filtration rate IFR within a capillary hemodialyzer. The method only requires dimensions of the hemodialyzer, dimensions well as physical parameters of the hollow fiber membranes present in the hemodialyzer, and flow rates of blood and dialysate through the hemodialyzer, all of which are readily available.

### BRIEF DESCRIPTION OF THE DRAWINGS

FIG. 1 shows a schematic perspective view and sectional detail views of a capillary hemodialyzer and fluid flows within the hemodialyzer;

FIG. 2 shows a comparison of experimentally determined internal filtration rates and internal filtration rates determined according to the method of the present disclosure for one model of a hemodialyzer;

FIG. 3 shows a comparison of experimentally determined internal filtration rates and internal filtration rates determined according to the method of the present disclosure for another model of a hemodialyzer.

### DETAILED DESCRIPTION

The present disclosure provides a computer-implemented method for determining an internal filtration rate IFR within a capillary hemodialyzer. The method comprises
i) acquiring, using a computer, data on physical properties of the hemodialyzer and of hollow fibers present in the hemodialyzer;
ii) acquiring, using the computer, a blood flow rate Q_{B} and a dialysis flow rate Q_{D} through the hemodialyzer during operation of the hemodialyzer;
iii) determining, using the computer, the internal filtration rate IFR of the hemodialyzer, based on the data acquired in steps i and ii.

In one embodiment, the method additionally comprises
iv) acquiring, using the computer, data on a duration TD of the operation of the hemodialyzer;
v) determining, using the computer, a total volume Vₜₒₜ of fluid exchanged through the wall of the hollow fiber membranes present in the hemodialyzer during operation of the hemodialyzer, based on the internal filtration rate IFR and the data acquired in step iv.

In a particular embodiment, data on physical properties of the hemodialyzer and of hollow fibers present in the hemodialyzer are acquired from a database in operative association with a processor of the computer.

In one embodiment of the method, the database comprises data on physical properties of a plurality of different hemodialyzers and of hollow fibers present in the hemodialyzers. For each hemodialyzer of the plurality of different hemodialyzers, such data may comprise a diameter d_{H} of the housing of the hemodialyzer, a number N of the hollow fibers present in the hemodialyzer, an effective length L of the hollow fibers present in the hemodialyzer, a total surface area Aₜₒₜ of the hollow fibers present in the hemodialyzer, an internal diameter d_{B} of the hollow fibers present in the hemodialyzer, a wall thickness δ_{M} of the hollow fibers present in the hemodialyzer, a porosity ε_{M} of the hollow fibers present in the hemodialyzer, and an ultrafiltration coefficient K_{UF} of the hollow fibers present in the hemodialyzer.

Additionally, the database may comprise data on a blood viscosity μ_{B} and a dialysate viscosity μ_{D}.

In a further embodiment, data on physical properties of the hemodialyzer and of hollow fibers present in the hemodialyzer and/or the blood flow rate Q_{B} and the dialysis flow rate Q_{D} through the hemodialyzer during operation of the hemodialyzer is acquired from an input device in operative association with a processor of the computer. The hemodialyzer is perfused with blood at a blood flow rate Q_{B} and dialysate at a dialysate flow rate Q_{D} during operation, i.e., when it is used in an external blood circuit to remove toxins from blood. The internal filtration rate IFR and the total volume Vₜₒₜ of fluid exchanged through the wall of the hollow fiber membranes present in the hemodialyzer vary with blood flow rate Q_{B} and dialysate flow rate Q_{D} through the hemodialyzer.

In one embodiment, the input device comprises a contactless reader. In a particular embodiment, the contactless reader is an optical reader which acquires data of a barcode or QR code present on a hemodialyzer. In another particular embodiment, the contactless reader is a sensor which acquires data from an RFID or NFC tag present on or in the hemodialyzer.

In a further embodiment, the input device comprises at least one user interface. In a particular embodiment, the user interface comprises a keyboard or a touchscreen. In a further embodiment of the system, the input device comprises a graphical use interface (GUI). In a particular embodiment, the input device comprises a touchscreen of a smartphone. The input device is used for manually entering data on physical properties of the hemodialyzer and of hollow fibers present in the hemodialyzer, and/or the blood flow rate Q_{B} and the dialysis flow rate Q_{D} through the hemodialyzer during operation of the hemodialyzer.

In another embodiment, the input device comprises an extracorporeal blood treatment apparatus. The extracorporeal blood treatment apparatus controls the parameters of an extracorporeal blood circuit comprising a hemodialyzer during operation, i.e. during the treatment of a patient. When used as an input device for the method of the present disclosure, the extracorporeal blood treatment apparatus provides data on physical properties of the hemodialyzer and of hollow fibers present in the hemodialyzer, and/or the blood flow rate Q_{B} and the dialysis flow rate Q_{D} through the hemodialyzer during operation of the hemodialyzer. In one embodiment of the method, the processor acquires real-time data of the blood flow rate Q_{B} and the dialysis flow rate Q_{D} through a hemodialyzer during operation of the hemodialyzer and, optionally, data on the duration TD of the operation, from the extracorporeal blood treatment apparatus, i.e. during an actual treatment. In another embodiment, a blood flow rate Q_{B} and a dialysis flow rate Q_{D} through a hemodialyzer during operation of the hemodialyzer and, optionally, a duration TD of the operation, are manually entered through the input device to simulate a treatment.

In one embodiment of the method, the data on physical properties of the hemodialyzer and of hollow fibers present in the hemodialyzer comprise a diameter d_{H} of the housing of the hemodialyzer, a number N of the hollow fibers present in the hemodialyzer, an effective length L of the hollow fibers present in the hemodialyzer, a total surface area Aₜₒₜ of the hollow fibers present in the hemodialyzer, an internal diameter d_{B} of the hollow fibers present in the hemodialyzer, a wall thickness δ_{M} of the hollow fibers present in the hemodialyzer, a porosity ε_{M} of the hollow fibers present in the hemodialyzer, and an ultrafiltration coefficient K_{UF} of the hollow fibers present in the hemodialyzer.

In one embodiment of the method, the internal filtration rate IFR is determined according to

$$IFR = N π {d}_{B} {\int }_{0}^{{x}_{i}} {J}_{v} ( z ) d z$$

with
N number of hollow fibers present in the hemodialyzer;
d_{B} internal diameter of hollow fibers present in the hemodialyzer;
Jᵥ(z) ultrafiltration flux through the membrane wall;
xᵢ location of inversion point (Jᵥ(xᵢ)=0).

This equation can be rewritten as

$$IFR = N π {d}_{B} K {\int }_{0}^{{x}_{i}} ( {P}_{B} ( z ) - {P}_{D} ( z ) - {π}_{o} ) d z$$

with
K

$$- \frac{{K}_{UF}}{{A}_{tot} {ε}_{M}} ,$$

K_{UF} ultrafiltration coefficient of the hollow fibers present in the hemodialyzer,
Aₜₒₜ total membrane area of the hollow fibers present in the hemodialyzer (Aₜₒₜ=Nπ_{B}L),
ε_{M} membrane porosity of the hollow fibers present in the hemodialyzer;
μ_{B}(Z) blood pressure;
μ_{D}(z) dialysate pressure;
Πₒ average oncotic pressure;

In another embodiment of the method, the internal filtration rate IFR is determined according to

$$IFR = N π {d}_{B} K \{ ( {P}_{B , out} - {P}_{D , out} - {π}_{0} ) {x}_{i} - \frac{128 {μ}_{B}}{N π {d}_{B}^{4}} [ {Q}_{B} ( \frac{{x}_{i}^{2}}{2} - L {x}_{i} ) - N π {d}_{B} [ \frac{{ν}_{2} - {ν}_{1}}{6 L} ( \frac{{x}_{i}^{4}}{4} - {L}^{3} {x}_{i} ) + \frac{{ν}_{1}}{2} ( \frac{{x}_{i}^{3}}{3} - {L}^{2} {x}_{i} ) ] ] + \frac{{μ}_{D}}{2 N π {f}_{{R}_{o} , {R}_{k}}} [ {Q}_{D} \frac{{x}_{i}^{2}}{2} - N π {d}_{B} [ \frac{{ν}_{2} - {ν}_{1}}{2 L} ( \frac{{x}_{i}^{4}}{12} - \frac{{L}^{2}}{2} {x}_{i}^{2} ) + {ν}_{1} ( \frac{{x}_{i}^{3}}{6} - \frac{L}{2} {x}_{i}^{2} ) ] ] \}$$

with
N number of hollow fibers present in the hemodialyzer;
d_{B} internal diameter of the hollow fibers present in the hemodialyzer;
K

$$- \frac{{K}_{UF}}{{A}_{tot} {ε}_{M}} ,$$

K_{UF} ultrafiltration coefficient of the hollow fibers present in the hemodialyzer,
Aₜₒₜ total membrane area of the hollow fibers present in the hemodialyzer (Aₜₒₜ=Nπd_{B}L),
ε_{M} membrane porosity of the hollow fibers present in the hemodialyzer;
μ_{B,out} blood pressure at hemodialyzer exit;
μ_{D,out} dialysate pressure at hemodialyzer exit;
Πₒ average oncotic pressure;
xᵢ location of inversion point (Jᵥ(xᵢ)=0);
μ_{B} blood viscosity;
Q_{B} blood flow rate;
L effective length of hollow fibers present in the hemodialyzer;
ν₁ Jᵥ(0);
ν₂ Jᵥ(L);
μ_{D} dialysate viscosity;
Q_{D} dialysate flow rate;

$${f}_{{R}_{o} , {R}_{k}} \frac{{R}_{k}^{4} - {R}_{o}^{4}}{16} + \frac{{R}_{k}^{2}}{2} ( \frac{{R}_{k}^{2}}{2} ln ( \frac{{R}_{o}}{{R}_{k}} ) + \frac{{R}_{k}^{2} - {R}_{o}^{2}}{4} ) - \frac{{R}_{o}^{2}}{8} ( {R}_{k}^{2} - {R}_{o}^{2} ) ,$$

$${R}_{o} = \frac{{d}_{B}}{2} + {δ}_{M} ,$$

$${R}_{k} = {R}_{o} + {δ}_{ε} ,$$

d_{B} internal diameter of hollow fibers present in the hemodialyzer,
δ_{M} wall thickness of the hollow fibers present in the hemodialyzer,
δ_{c} half the distance between adjacent hollow fibers present in the hemodialyzer.

In a further embodiment of the method, some of the parameters in the above equation for IFR are calculated according to the following equations:

$$1. {f}_{{R}_{o} , {R}_{k}} = \frac{{R}_{k}^{4} - {R}_{o}^{4}}{16} + \frac{{R}_{k}^{2}}{2} ( \frac{{R}_{k}^{2}}{2} ln ( \frac{{R}_{o}}{{R}_{k}} ) + \frac{{R}_{k}^{2} - {R}_{0}^{2}}{4} ) - \frac{{R}_{o}^{2}}{8} ( {R}_{k}^{2} - {R}_{o}^{2} )$$

$$2. K = - \frac{{K}_{UF}}{{A}_{tot} {ε}_{M}}$$

$$3. {E}_{1} = \frac{{μ}_{D} {d}_{B} {L}^{3}}{16 {f}_{{R}_{o} , {R}_{k}}} - \frac{80 {μ}_{B} {L}^{3}}{3 {d}_{B}^{3}}$$

$$4. {E}_{2} = \frac{5 {μ}_{D} {d}_{B} {L}^{3}}{48 {f}_{{R}_{o} , {R}_{k}}} - \frac{16 {μ}_{B} {L}^{3}}{{d}_{B}^{3}}$$

$$5. {R}_{1} = 1 + \frac{128}{3} K \frac{{μ}_{B} {L}^{2}}{{d}_{B}^{3}}$$

$$6. {R}_{2} = 1 - K \frac{{μ}_{D} {d}_{B} {L}^{2}}{6 {f}_{{R}_{o} , {R}_{k}}} + \frac{16}{9} {K}^{2} \frac{{μ}_{B} {μ}_{D} {L}^{4}}{{R}_{1} {f}_{{R}_{o} , {R}_{k}} {d}_{B}^{2}}$$

$$7. {F}_{1} = ( {P}_{B , o u t} - {π}_{0} ) L + 64 \frac{{μ}_{B} {L}^{2} {Q}_{B}}{N π {d}_{B}^{4}} + \frac{{μ}_{D} {L}^{2} {Q}_{D}}{4 N π {f}_{{R}_{0} , {R}_{k}}}$$

$$8. {F}_{2} = {E}_{2} - \frac{64}{3} \frac{{E}_{1} {μ}_{B} {L}^{2} K}{{R}_{1} {d}_{B}^{3}}$$

$$9. {B}_{1} = {P}_{B , o u t} - {P}_{D , o u t} - {π}_{o} + \frac{128 {μ}_{B} {Q}_{B} L}{N π {d}_{B}^{4}}$$

$$10. {B}_{2} = {P}_{B , o u t} - {π}_{o} + \frac{128 {μ}_{B} {Q}_{B} L}{N π {d}_{B}^{4}}$$

$$11. {D}_{1} = {P}_{B , o u t} - {P}_{D , o u t} - {π}_{o} + \frac{{μ}_{D} {Q}_{D} L}{2 N π {f}_{{R}_{o} , {R}_{k}}}$$

$$12. {D}_{2} = {P}_{B , o u t} - {π}_{o} + \frac{{μ}_{D} {Q}_{D} L}{2 N π {f}_{{R}_{o} , {R}_{k}}}$$

$$13. {G}_{1} = {F}_{1} + \frac{{E}_{1}}{{R}_{1}} K {B}_{2} + \frac{{F}_{2}}{{R}_{2}} K {D}_{2} + \frac{{F}_{2}}{{R}_{2}} {K}^{2} \frac{{μ}_{D} {d}_{B} {L}^{2}}{12 {f}_{{R}_{o} , {R}_{k}}} {B}_{2}$$

$$14. {G}_{2} = L + \frac{{E}_{1}}{{R}_{1}} K + \frac{{F}_{2}}{{R}_{2}} K + \frac{{F}_{2}}{{R}_{2}} {K}^{2} \frac{{μ}_{D} {d}_{B} {L}^{2}}{12 {R}_{1} {f}_{{R}_{o} , {R}_{k}}}$$

$$15. {P}_{D , o u t} = \frac{1}{{G}_{2}} ( {G}_{1} - \frac{U F}{N π {d}_{B} K} )$$

$$16. {v}_{1} = {J}_{v} 0 ) = K \frac{1}{{R}_{1}} ( {B}_{1} - \frac{64 {μ}_{B} {L}^{2}}{3 {d}_{B}^{3}} {v}_{2} )$$

$$17. {v}_{2} = {J}_{v} ( L ) = \frac{K}{{R}_{2}} ( {D}_{1} + K \frac{{μ}_{D} {d}_{B} {L}^{2} {B}_{1}}{12 {R}_{1} {f}_{{R}_{o} , {R}_{k}}} )$$

$$18. {x}_{i} = \frac{{v}_{1}}{{v}_{1} - {v}_{2}}$$

with
N number of hollow fibers present in the hemodialyzer;
L effective length of the hollow fibers present in the hemodialyzer;
d_{B} internal diameter of the hollow fibers present in the hemodialyzer;
Aₜₒₜ total membrane area of the hollow fibers present in the hemodialyzer (Aₜₒₜ=Nπd_{B}L);
UF net ultrafiltration flow rate;
K_{UF} UF coefficient;
Q_{B} blood flow rate;
Q_{D} dialysate flow rate;
μ_{B,out} blood pressure at dialyzer exit;
μ_{D,out} dialysate pressure at dialyzer exit;
πₒ average oncotic pressure;
μ_{B} blood viscosity;
μ_{D} dialysate viscosity;
ε_{M} membrane porosity;
δ_{M} membrane thickness.

In one embodiment of the method, the total volume Vₜₒₜ of fluid exchanged through the wall of the hollow fiber membranes present in the hemodialyzer over a time period TD of operation of the hemodialyzer is determined according to

$${V}_{tot} = {\int }_{0}^{T D} IFR ( t ) dt$$

The present disclosure also provides a system comprising
a) a database comprising data on physical properties of a plurality of capillary hemodialyzers and of hollow fibers present therein; and/or
b) an input device configured for providing data on physical properties of a capillary hemodialyzer and of hollow fibers present in the hemodialyzer and/or for providing a blood flow rate and a dialysis flow rate through the hemodialyzer during operation of the hemodialyzer;
c) an output device configured for output of data received from a computer processor in operative association with the output device;
d) a computer processor programmed for communication with the database and/or the input device, and for communication with the output device, the processor programmed for
   a. acquiring data from the database and/or the input device,
   b. determining an internal flow rate IFR within a capillary hemodialyzer, based on the acquired data,
   c. optionally, determining a total volume Vₜₒₜ of fluid exchanged through the wall of the hollow fiber membranes present in the hemodialyzer during operation of the hemodialyzer;
   d. transmitting the determined internal flow rate IFR, and, optionally, the total volume Vₜₒₜ of fluid exchanged to the output device.

In one embodiment of the system, the input device, the output device, and the processor are contained in a portable device. In one embodiment, the portable device is a portable computer, for instance, a laptop, a tablet computer, or a PDA. In a further embodiment, the portable device is a mobile communication device, for instance, a smartphone.

In one embodiment of the system, the output device is a display device. Examples of suitable display devices include monitors, computer displays, and touchscreens. In a particular embodiment, the display device is a touchscreen of a smartphone.

In one embodiment of the system, the database is present in a computer memory in operative association with the computer processor. In a further embodiment, the computer memory is contained in a portable device comprising the input device, the output device, and the processor. In another embodiment, the database is present in a remote computer memory, a network drive, or a cloud memory accessible via the internet.

The present disclosure also provides a computer program for instructing a computer processor to perform the method of
a. acquiring data from a database and/or an input device in operative association with the processor;
b. determining an internal filtration rate IFR within a capillary hemodialyzer, based on the acquired data;
c. optionally, determining a total volume Vₜₒₜ of fluid exchanged through the wall of the hollow fiber membranes present in the hemodialyzer during operation of the hemodialyzer;
d. transmitting the determined internal flow rate IFR, and, optionally, the total volume Vₜₒₜ of fluid exchanged to an output device in operative association with the processor.

In one embodiment, the computer program takes the form of a software application (“app”) that can be installed and run on a smartphone.

The present disclosure also provides a non-transitory computer-readable medium comprising the computer program.

It will be understood that the features mentioned above and those described hereinafter can be used not only in the combination specified but also in other combinations or on their own, without departing from the scope of the present invention.

The method of the present disclosure will now be further described in the following examples and referring to the attached drawings.

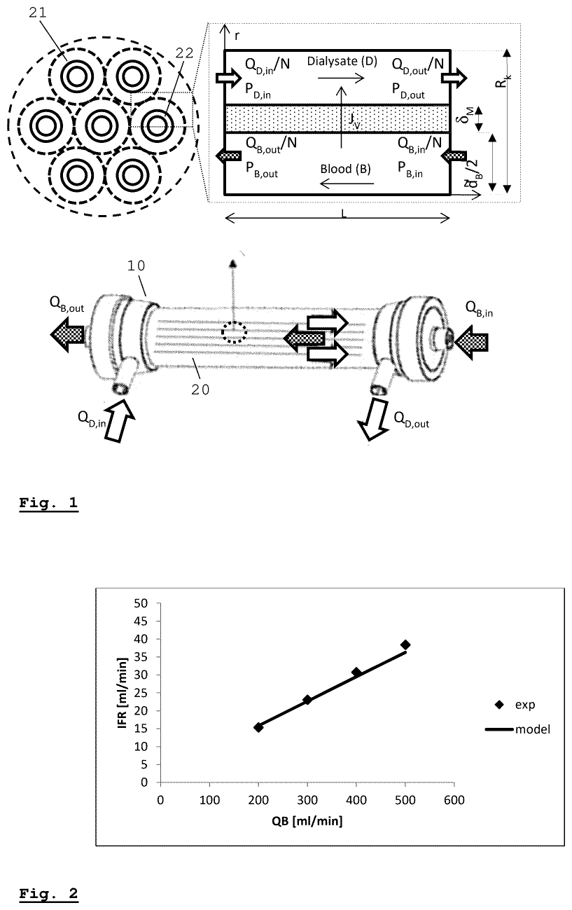

FIG. 1 shows a schematic perspective view and sectional detail views of a capillary hemodialyzer 10 and fluid flows within the hemodialyzer 10. The hemodialyzer 10 comprises a bundle 20 of hollow fiber membranes 21. In the top left corner of FIG. 1, a detail of a cross-section of the bundle 20 is shown with several hollow fiber membranes 21, each having a membrane wall 22. Blood flow and dialysate flow through the hemodialyzer 10 are indicated by arrows. Blood enters the hemodialyzer 10 through a blood inlet located on a header of the hemodialyzer 10 at a blood flow rate Q_{B,in}, flows through the lumen of the hollow fiber membranes 21 of the bundle 20, and leaves the hemodialyzer 10 at a blood flow rate Q_{B,out} through a blood outlet located on another header of the hemodialyzer 10. Dialysate enters the hemodialyzer 10 through a dialysate inlet positioned near one end of the hemodialyzer 10 at a dialysate flow rate Q_{D,in}, flows through the space outside the hollow fiber membranes 21 of the bundle 20, and leaves the hemodialyzer 10 at a dialysate flow rate Q_{D,out} through a dialysate outlet positioned near the opposite end of the hemodialyzer 10.

In the top right corner of FIG. 1, a detail of a longitudinal section of one of hollow fiber membranes 21 is shown.

The effective length of the hollow fiber membrane 21 is L, the internal radius of the hollow fiber membrane 21 is d_{B}/2, the thickness of the membrane wall 22 is δ_{M}, the distance between the longitudinal axis of one hollow fiber membrane 21 and the longitudinal axis of an adjacent hollow fiber membrane 21 is 2*Rₖ, with Rₖ=Rₒ+δ_{c}, or Rₖ=d_{B}/2+δ_{M}+δ_{c}, Rₒ being half the outside diameter of the hollow fiber membrane 21, and δ_{c} being half the distance between two adjacent hollow fiber membranes 21. Blood flows in the lumen of the hollow fiber membrane 21, dialysate flows on the outside of the hollow fiber membrane 21, and ultrafiltration flux Jᵥ permeates the membrane wall 22.

### Example 1

Internal filtration rates within a hemodialyzer (Polyflux® 210H, Gambro Dialysatoren GmbH, 72379 Hechingen, Germany) were determined according to one embodiment of the method of the present disclosure, and compared to internal flow rates determined experimentally (from D. Schneditz et al.: “Internal filtration, filtration fraction, and blood flow resistance in high- and low-flux dialyzers”, Clin. Hemorheol. Microcirc. 58 (2014) 455-469).

The following physical properties of the hemodialyzer were used for the determination:
hollow fiber inner diameter d_{B}=215 μm
Membrane wall thickness δ_{M}=50 μm
Membrane porosity ε_{M}=0.7
Number of fibers N=11,640
Effective length L=270 mm
Ultrafiltration coefficient K_{UF}=85 ml/(h*mm Hg)
Half distance between adjacent fibers δ_{c}=68.2 μm
Hemodialyzer housing inner diameter d_{H}=48.7 mm

Values for blood viscosity of μ_{B}=5.2 mPas and dialysate viscosity of μ_{D}=0.96 mPas were used.

Internal filtration rate IFR was determined for a dialysis flow rate Q_{D} of 500 ml/min, and blood flow rates Q_{B} of 200 ml/min, 300 ml/min, 400 ml/min, and 500 ml/min, respectively.

The values for the following constant parameters were obtained as described above.

$$K = 0.119 {μm}^{2} s / kg$$

$${R}_{1} = 1.19$$

$${R}_{2} = 1.01$$

$${E}_{1} = - 285 MPas$$

$${E}_{2} = - 182 MPas$$

$${F}_{2} = - 159 MPas$$

$${G}_{2} = 0.2 \; 23 m$$

The following table shows IFR at Q_{D}=500 ml/min and different blood flow rates Q_{B}.

| Q_{B} [ml/min] | 200 | 300 | 400 | 500 |
|---|---|---|---|---|
| IFR [ml/min] | 15.4 | 23.1 | 30.7 | 38.4 |

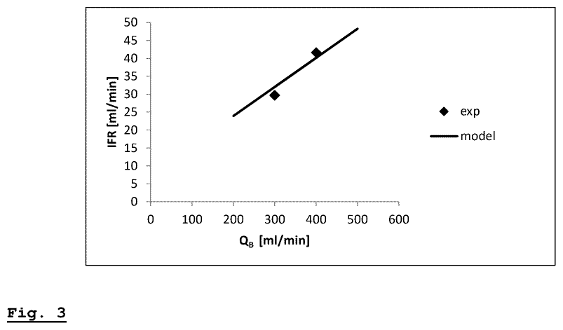

FIG. 2 shows a comparison of the internal flow rates ° determined according to the method of the present disclosure and experimentally determined internal flow rates taken from D. Schneditz et al.: “Internal filtration, filtration fraction, and blood flow resistance in high- and low-flux dialyzers”, Clin. Hemorheol. Microcirc. 58 (2014) 455-469.

### Example 2

Internal filtration rates within a hemodialyzer (Theranova® 400, Gambro Dialysatoren GmbH, 72379 Hechingen, Germany) were determined according to one embodiment of the method of the present disclosure, and compared to internal flow rates determined experimentally (from A. Lorenzin et al.: “Quantification of Internal Filtration in Hollow Fiber Hemodialyzers with Medium Cut-off Membrane”, Blood Purif. 46 (2018) 196-204).

The following physical properties of the hemodialyzer were used for the determination:
hollow fiber inner diameter d_{B}=180 μm
Membrane wall thickness δ_{M}=35 μm
Membrane porosity ε_{M}=0.5
Number of fibers N=13,000
Effective length L=236 mm
Ultrafiltration coefficient K_{UF}=48 ml/(h*mm Hg)
half distance between adjacent fibers δ_{c}=41.9 μm
Hemodialyzer housing inner diameter d_{H}=38 mm

Values for blood viscosity of μ_{B}=5.0 mPas and dialysate viscosity of μ_{D}=0.96 mPas were used.

Internal filtration rate IFR was determined for a dialysis flow rate Q_{D} of 500 ml/min, and blood flow rates Q_{B} of 300 ml/min and 400 ml/min, respectively.

The values for the following constant parameters were obtained as described above.

$$K = 0.116 {μm}^{2} s / kg$$

$${R}_{1} = 1.24$$

$${R}_{2} = 1.04$$

$${E}_{1} = - 335 MPas$$

$${E}_{2} = - 237 MPas$$

$${F}_{2} = - 206 MPas$$

$${G}_{2} = 0.1 \; 82 m$$

The following table shows IFR at Q_{D}=500 ml/min and different blood flow rates Q_{B}.

| Q_{B} [ml/min] | 300 | 400 |
|---|---|---|
| IFR [ml/min] | 26.7 | 41.6 |

FIG. 3 shows a comparison of the internal flow rates determined according to the method of the present disclosure and experimentally determined internal flow rates taken from A. Lorenzin et al.: “Quantification of Internal Filtration in Hollow Fiber Hemodialyzers with Medium Cut-off Membrane”, Blood Purif. 46 (2018) 196-204.

### LIST OF REFERENCE SIGNS

10 hemodialyzer
20 bundle of hollow fiber membranes
21 hollow fiber membrane
22 membrane wall
L length of hollow fiber membrane
δ_{M} thickness of membrane wall
d_{B} internal diameter of hollow fiber membrane
Rₖ radius of space occupied by hollow fiber membrane

1. A method of performing expanded hemodialysis (HDx) on a patient, the method comprising a step of using a capillary hemodialyzer on the patient,
wherein the capillary hemodialyzer comprises a housing,
wherein the capillary hemodialyzer comprises hollow fiber membranes,
wherein the capillary hemodialyzer is configured to comprise an internal filtration rate (IFR),
wherein the capillary hemodialyzer comprises properties including dimensions, a flow rate of blood through the capillary hemodialyzer, and a flow rate of dialysate through the capillary hemodialyzer,
wherein the hollow fiber membranes comprise properties including dimensions and physical parameters, and
wherein the method of performing HDx on the patient comprises a step of measuring the IFR of the capillary hemodialyzer, wherein the IFR is calculated according to steps comprising:
i) acquiring data on the properties of the capillary hemodialyzer and the properties of hollow fiber membranes present in the capillary hemodialyzer, the data comprising a diameter d_{H} of the housing of the capillary hemodialyzer, a number N of the hollow fiber membranes present in the capillary hemodialyzer, an effective length L of the hollow fiber membranes present in the capillary hemodialyzer, a total surface area Aₜₒₜ of the hollow fiber membranes present in the capillary hemodialyzer, an internal diameter d_{B} of the hollow fiber membranes present in the capillary hemodialyzer, a wall thickness δ_{M} of the hollow fiber membranes present in the capillary hemodialyzer, a porosity ε_{M} of the hollow fiber membranes present in the capillary hemodialyzer, and an ultrafiltration coefficient K_{UF} of the hollow fiber membranes present in the capillary hemodialyzer;
ii) acquiring a blood flow rate Q_{B} and a dialysis flow rate Q_{D} through the capillary hemodialyzer during operation of the capillary hemodialyzer;
iii) determining the IFR of the capillary hemodialyzer, based on the data acquired in steps i and ii, wherein the IFR is determined according to
$$IFR = N π {d}_{B} K \{ ( {P}_{B , out} - {P}_{D , out} - {π}_{0} ) {x}_{i} - \frac{128 {μ}_{B}}{N π {d}_{B}^{4}} [ {Q}_{B} ( \frac{{x}_{i}^{2}}{2} - L {x}_{i} ) - N π {d}_{B} [ \frac{{v}_{2} - {v}_{1}}{6 L} ( \frac{{x}_{i}^{4}}{4} - {L}^{3} {x}_{i} ) + \frac{{v}_{1}}{2} ( \frac{{x}_{i}^{3}}{3} - {L}^{2} {x}_{i} ) ] ] + \frac{{μ}_{D}}{2 N π {f}_{{R}_{O} {R}_{K}}} [ {Q}_{D} \frac{{x}_{i}^{2}}{2} - Nπ {d}_{B} [ \frac{{v}_{2} - {v}_{1}}{2 L} ( \frac{{x}_{i}^{4}}{12} - \frac{{L}^{2}}{2} {x}_{i}^{2} ) + {v}_{1} ( \frac{{x}_{i}^{3}}{6} - \frac{L}{2} {x}_{i}^{2} ) ] ] \}$$
wherein
N refers to the number of hollow fiber membranes present in the capillary hemodialyzer;
d_{B} refers to the internal diameter of hollow fiber membranes present in the capillary hemodialyzer;
K refers to
$$\frac{{K}_{UF}}{{A}_{tot} {E}_{M}}$$
in which K_{UF} refers to the ultrafiltration coefficient of the hollow fiber membranes present in the capillary hemodialyzer,
Aₜₒₜ refers to the total membrane area of the hollow fiber membranes present in the capillary hemodialyzer
$$( {A}_{tot} = N Π {d}_{B} L ) ,$$
ε_{M} refers to the membrane porosity of the hollow fiber membranes present in the capillary hemodialyzer;
μ_{B}, out refers to the blood pressure at capillary hemodialyzer exit;
μ_{D}, out refers to the dialysate pressure at capillary hemodialyzer exit;
Πₒ refers to the average oncotic pressure;
xᵢ refers to the location of inversion point (J_{ν}(xᵢ)=0);
μ_{B} refers to the blood viscosity;
Q_{B} refers to the blood flow rate;
L refers to the effective length of hollow fiber membranes present in the capillary hemodialyzer;
ν₁ refers to J(0);
ν₂ refers to Jᵥ(L);
μ_{D} refers to the dialysate viscosity;
Q_{D} refers to the dialysate flow rate; and
$${f}_{{R}_{O} , {R}_{K}} = \frac{{R}_{K}^{4} - {R}_{O}^{4}}{16} + \frac{{R}_{K}^{2}}{2} ( \frac{{R}_{K}^{2}}{2} ln ( \frac{{R}_{O}}{{R}_{K}} ) + \frac{{R}_{K}^{2} - {R}_{O}^{2}}{4} ) - \frac{{R}_{O}^{2}}{8} ( {R}_{K}^{2} - {R}_{O}^{2} ) ,$$
$${R}_{O} = \frac{{d}_{B}}{2} + {δ}_{M}$$
$${R}_{K} = {R}_{O} + {δ}_{ε}$$
wherein d_{B} refers to the internal diameter of the hollow fiber membranes present in the h capillary hemodialyzer, δ_{M} refers to the wall thickness of the hollow fiber membranes present in the capillary hemodialyzer, δ_{c} refers to the thickness of the diffusion layer around the hollow fiber membranes present in the capillary hemodialyzer.

2. The method of claim 1, additionally comprising the steps of:
iv) acquiring, using the computer, data on a duration TD of the operation of the hemodialyzer; and
v) determining, using the computer, a total volume Vₜₒₜ of fluid exchanged through the wall of the hollow fiber membranes present in the capillary hemodialyzer during operation of the capillary hemodialyzer, based on the IFR and the data acquired in step iv, wherein the total volume Vₜₒₜ is determined according to
$${V}_{tot} = {\int }_{0}^{T D} IFR ( t ) dt .$$

3. The method of claim 1, wherein data on physical properties of the capillary hemodialyzer and of hollow fiber membranes present in the capillary hemodialyzer are acquired from a database in operative association with a processor of the computer.

4. The method of claim 1, wherein data on physical properties of the capillary hemodialyzer and of hollow fiber membranes present in the capillary hemodialyzer and/or the blood flow rate through the capillary hemodialyzer and the dialysis flow rate through the capillary hemodialyzer during operation of the capillary hemodialyzer is acquired from an input device in operative association with a processor of the computer.

5. The method of claim 4, wherein the input device comprises a contactless reader.

6. The method of claim 4, wherein the input device comprises at least one user interface.

7. The method of claim 4, wherein the input device comprises an extracorporeal blood treatment apparatus.

8. The method of claim 1, wherein the IFR is calculated using a computer-implemented method.

9. The method of claim 1, wherein the calculated IFR is used to configure the hollow fiber membranes in the capillary hemodialyzer to increase convective transport of solutes across the hollow fiber membranes.

10. The method of claim 1, wherein the calculated IFR is used to configure the hollow fiber membranes in the capillary hemodialyzer to adjust an amount of back filtration in the capillary hemodialyzer.

11. The method of claim 1, wherein the hollow fiber membranes comprise a medium cut-off (MCO) membrane.

12. A method of performing expanded hemodialysis (HDx) on a patient, the method comprising a step of using a capillary hemodialyzer on the patient,
wherein the capillary hemodialyzer comprises a housing,
wherein the capillary hemodialyzer comprises hollow fiber membranes,
wherein the capillary hemodialyzer is configured to comprise an internal filtration rate (IFR),
wherein the capillary hemodialyzer comprises properties including dimensions, a flow rate of blood through the capillary hemodialyzer, and a flow rate of dialysate through the capillary hemodialyzer,
wherein the hollow fiber membranes comprise properties including dimensions and physical parameters, and
wherein the method of performing HDx on the patient comprises a step of increasing the IFR of the capillary hemodialyzer, wherein the IFR is calculated according to steps comprising:
i) acquiring data on the properties of the capillary hemodialyzer and the properties of hollow fiber membranes present in the capillary hemodialyzer, the data comprising a diameter d_{H} of the housing of the capillary hemodialyzer, a number N of the hollow fiber membranes present in the capillary hemodialyzer, an effective length L of the hollow fiber membranes present in the capillary hemodialyzer, a total surface area Aₜₒₜ of the hollow fiber membranes present in the capillary hemodialyzer, an internal diameter d_{B} of the hollow fiber membranes present in the capillary hemodialyzer, a wall thickness δ_{M} of the hollow fiber membranes present in the capillary hemodialyzer, a porosity ε_{M} of the hollow fiber membranes present in the capillary hemodialyzer, and an ultrafiltration coefficient K_{UF} of the hollow fiber membranes present in the capillary hemodialyzer;
ii) acquiring a blood flow rate Q_{B} and a dialysis flow rate Q_{D} through the capillary hemodialyzer during operation of the capillary hemodialyzer;
iii) determining the IFR of the capillary hemodialyzer, based on the data acquired in steps i and ii, wherein the IFR is determined according to
$$IFR = N π {d}_{B} K \{ ( {P}_{B , out} - {P}_{D , out} - {π}_{0} ) {x}_{i} - \frac{128 {μ}_{B}}{N π {d}_{B}^{4}} [ {Q}_{B} ( \frac{{x}_{i}^{2}}{2} - L {x}_{i} ) - N π {d}_{B} [ \frac{{v}_{2} - {v}_{1}}{6 L} ( \frac{{x}_{i}^{4}}{4} - {L}^{3} {x}_{i} ) + \frac{{v}_{1}}{2} ( \frac{{x}_{i}^{3}}{3} - {L}^{2} {x}_{i} ) ] ] + \frac{{μ}_{D}}{2 N π {f}_{{R}_{O} , {R}_{K}}} [ {Q}_{D} \frac{{x}_{i}^{2}}{2} - N π {d}_{B} [ \frac{{v}_{2} - {v}_{1}}{2 L} ( \frac{{x}_{i}^{4}}{12} - \frac{{L}^{2}}{2} {x}_{i}^{2} ) + {v}_{1} ( \frac{{x}_{i}^{3}}{6} - \frac{L}{2} {x}_{i}^{2} ) ] ] \}$$
wherein
N refers to the number of hollow fiber membranes present in the capillary hemodialyzer;
d_{B} refers to the internal diameter of hollow fiber membranes present in the capillary hemodialyzer;
K refers to
$$\frac{{K}_{UF}}{{A}_{tot} {E}_{M}}$$
in which K_{UF} refers to the ultrafiltration coefficient of the hollow fiber membranes present in the capillary hemodialyzer,
Aₜₒₜ refers to the total membrane area of the hollow fiber membranes present in the capillary hemodialyzer
$$( {A}_{tot} = N Π {d}_{B} L ) ,$$
ε_{M} refers to the membrane porosity of the hollow fiber membranes present in the capillary hemodialyzer;
P_{B, out} refers to the blood pressure at capillary hemodialyzer exit;
P_{D, out} refers to the dialysate pressure at capillary hemodialyzer exit;
Πₒ refers to the average oncotic pressure;
xᵢ refers to the location of inversion point (J_{ν}(xᵢ)=0);
μ_{B} refers to the blood viscosity;
Q_{B} refers to the blood flow rate;
L refers to the effective length of hollow fiber membranes present in the capillary hemodialyzer;
ν₁ refers to J(0);
ν₂ refers to Jᵥ(L);
μ_{D} refers to the dialysate viscosity;
Q_{D} refers to the dialysate flow rate; and
$${f}_{{R}_{O} , {R}_{K}} = \frac{{R}_{K}^{4} - {R}_{O}^{4}}{16} + \frac{{R}_{K}^{2}}{2} ( \frac{{R}_{K}^{2}}{2} ln ( \frac{{R}_{O}}{{R}_{K}} ) + \frac{{R}_{K}^{2} - {R}_{O}^{2}}{4} ) - \frac{{R}_{O}^{2}}{8} ( {R}_{K}^{2} - {R}_{O}^{2} ) ,$$
$${R}_{O} = \frac{{d}_{B}}{2} + {δ}_{M}$$
$${R}_{K} = {R}_{O} + {δ}_{ε}$$
wherein d_{B} refers to the internal diameter of the hollow fiber membranes present in the h capillary hemodialyzer, δ_{M} refers to the wall thickness of the hollow fiber membranes present in the capillary hemodialyzer, δ_{c} refers to the thickness of the diffusion layer around the hollow fiber membranes present in the capillary hemodialyzer.

13. The method of claim 12, additionally comprising the steps of:
iv) acquiring, using the computer, data on a duration TD of the operation of the hemodialyzer; and
v) determining, using the computer, a total volume Vₜₒₜ of fluid exchanged through the wall of the hollow fiber membranes present in the capillary hemodialyzer during operation of the capillary hemodialyzer, based on the IFR and the data acquired in step iv, wherein the total volume Vₜₒₜ is determined according to
$${V}_{tot} = {\int }_{0}^{T D} IFR ( t ) dt .$$

14. The method of claim 12, wherein data on physical properties of the capillary hemodialyzer and of hollow fiber membranes present in the capillary hemodialyzer are acquired from a database in operative association with a processor of the computer.

15. The method of claim 12, wherein data on physical properties of the capillary hemodialyzer and of hollow fiber membranes present in the capillary hemodialyzer and/or the blood flow rate through the capillary hemodialyzer and the dialysis flow rate through the capillary hemodialyzer during operation of the capillary hemodialyzer is acquired from an input device in operative association with a processor of the computer.

16. The method of claim 15, wherein the input device comprises an extracorporeal blood treatment apparatus.

17. The method of claim 12, wherein the IFR is calculated using a computer-implemented method.

18. The method of claim 12, wherein the calculated IFR is used to configure the hollow fiber membranes in the capillary hemodialyzer to increase convective transport of solutes across the hollow fiber membranes.

19. The method of claim 12, wherein the calculated IFR is used to configure the hollow fiber membranes in the capillary hemodialyzer to adjust an amount of back filtration in the capillary hemodialyzer.

20. The method of claim 12, wherein the hollow fiber membranes comprise a medium cut-off (MCO) membrane.
